(19) United States
(12) Patent Application Publication
(10) Pub. No.: US 2004/0087520 A1
(43) Pub. Date: May 6, 2004

(54) Compositions and methods for delivery of therapeutic agents

(76) Inventors: Dipak K. Chowdhury, Lexington, KY (US); Mark S. Paxton, Lexington, KY (US); Santos B. Murty, Lexington, KY (US); B. Ram Murty, Lexington, KY (US)

Correspondence Address: Dr. Dipak K. Chowdhury, Murty Pharmaceuticals, Inc., 518 Codell Drive, Lexington, KY, 40509, US

(21) Appl. No.: 10/694,377

(22) Filed: Oct. 27, 2003

Related U.S. Application Data
(60) Provisional application No. 60/421,481, filed on Oct. 25, 2002.

(51) Int. Cl. A61K 31/704

(52) U.S. Cl. 514/35

(57) ABSTRACT

Disclosed are compositions for the delivery of therapeutic agents and methods of using these compositions.

### PRIORITY

Priority is claimed on the basis of provisional application No. 60/421,481, filed Oct. 25, 2002.

### STATEMENT REGARDING FEDERAL SPONSORSHIP

Not applicable

### FIELD OF THE INVENTION

The invention relates to compositions and methods for delivery of therapeutic agents

### BACKGROUND OF THE INVENTION

Surgical site infections can be problematic, risky, and at times expensive. It has been estimated that surgical site infections lead to an annual increased expenditure of $3.3 billion (measured in 1992 dollars) [Quinn, Francis B., et al., “Microbiology, Infections and Antibiotic Therapy,” Grand Rounds Presentation, UTMB Dept. of Otolaryngology, March 2000]. The need for lessening the probability of surgical site infection is reflected in reports concerning morbidity and mortality associated with arthroscopic knee surgery. At the time of preparation of the present application, links to web pages showing summaries of data concerning outpatient and inpatient surgeries can be found on the internet at www.cdc.gov/nchs/fastats.

Antibiotics have traditionally been delivered in different dosage forms for oral or parenteral administration. These traditional delivery forms may provide excellent results when used for therapy or treatment of an active systemic infection, but these traditional delivery forms are in general not as effective when utilized to prevent localized infection at a surgical site. This is because the problem addressed by traditional delivery forms of antibiotics for the treatment of active systemic infection may be distinct from the problem addressed by delivery forms of antibiotics for the prevention of localized infection at a surgical site.

To be effective at preventing surgical site infection, oral or parenteral antibiotics must in general be administered prior to bacterial contamination of the surgical site, which in general requires administration before the surgical procedure. Where used afterwards, there is in general no beneficial effect when oral or parenteral antibiotics are administered more than three hours after surgery. Furthermore, where oral or parenteral antibiotics are administered prior to surgery in order to prevent surgical site infection, administration is required for at least five days after surgery, although standard practice typically requires a postoperative course of ten days.

Certain studies establish that there are significant benefits in the form of reduced infection rates associated with localized application of antibiotics [Polk, Hiram C., et al., “Prophylactic Antibiotics in Surgery and Surgical Wound Infections,” Dept. of Surgery, University of Louisville, 2000, citing Bergamini et al., “Combined Topical and Systemic Antibiotic Prophylaxis in Experimental Wound Infection,” Am J Surg., 1984; 147:753-756]. For instance, antibiotic powders, pastes, and aqueous solutions rinsed into incisions prior to closure have been found to be more effective than oral or parenteral antibiotics at preventing surgical site infection. Similarly, it has been found that surgical site infection rates were significantly reduced when patients' incisions were rinsed with an antibiotic solution for three days following surgery.

Studies therefore establish that localized application of an antibiotic at a surgical site can prevent localized infection at that site. There is a recognized need in the medical community for a preventive antibiotic formulation that may be applied locally at a surgical site. In addition, there is a recognized need among veterinarians and dentists for a preventive antibiotic formulation that may be applied locally at a surgical site. It would also be beneficial to deliver directly to the surgical site an analgesic or a local anesthetic for pain management, or, more generally, to deliver directly to an arbitrary site in a vertebrate subject a therapeutic agent needed for the prevention, treatment, lessening or amelioration of a condition which it is desired to prevent, treat, lessen or ameliorate in the subject.

### DESCRIPTION OF THE INVENTION

The invention provides pharmaceutical compositions useful for application or delivery of therapeutic agents, and methods of using the compositions.

For example, a composition according to the invention is useful for application of a therapeutic agent to, or contact of an agent with, an exposed surgical wound. For example, a composition according to the invention provides a dosage form possessing a bioadhesive property (texture).

In an embodiment, a composition according to the invention possesses substantially greater viscosity at about 37 degrees Celsius than at about 20 degrees Celsius.

In an embodiment, a composition according to the invention provides a sustained-release dosage form for a therapeutic agent, such as an antibiotic or an analgesic.

When used in connection with the invention, a “therapeutic agent” refers to a composition used for (a) the treatment of, therapy of, prophylaxis of, lessening the severity of, amelioration of, or forestalling (b) an injury, a disease, an infection, discomfort, pain, or a malady in a vertebrate. For example, a therapeutic agent comprises a composition known in the art to be a drug.

In an embodiment, the invention provides a medicinal substance comprising a composition possessing a viscosity that, at least within a portion of a certain range of temperatures, increases as the temperature of the composition increases. In a preferred embodiment, the certain range of temperatures is from about 15 degrees Celsius below the body temperature of a vertebrate in which it is desired to deliver a therapeutic agent to about the body temperature of the vertebrate.

In a preferred embodiment, the invention provides a composition useful for the topical administration of a drug to the skin of a vertebrate to which it is desired to administer the drug.

For example, an analgesic drug for topical administration according to the invention is acetaminophen, tramadol, sodium salicylate and sodium aurothiomate.

For example, an antifungal drug for topical administration according to the invention is water-soluble salt of miconazole.

For example, an antiviral drug for topical administration according to the invention is acyclovir sodium, ganciclovir sodium and other sodium salts.

For example, an anesthetic drug for topical administration according to the invention is prilocaine hydrochloride or lidocaine hydrochloride.

For example, an antimicrobial drug for topical administration according to the invention is chlorhexidine gluconate.

For example, an antibacterial drug for topical administration according to the invention is a member of the group consisting of water-soluble beta-lactam antibiotics like benzyl penicillin, benzathine penicillin, cloxacillin sodium, piperacillin sodium, carbenicillin disodium; water soluble salts of cephalosporins like cefapirin sodium, cefalothin sodium, cefuroxime sodium, cefinenoxime hydrochloride, cefonicid sodium, cefoperazone sodium, cefotaxime sodium, cefotetan disodium, cefoxitin sodium and others, polypeptide antibiotics like bacitracin, polymyxin B sulfate, aminoglycoside antibiotics like gentamicin, vancomycin, neomycin sulfate; oxacillin sodium sulfate, nitrofurantoin sodium; tetracyclines like doxycycline sodium, doxyxcycline hydrochloride; the antimicrobial combination of fludalanine/pentizdone, mafenide acetate.

For example, an anti-inflammatory drug for topical administration according to the invention is a member of the group consisting of water soluble salts of corticosteroids like dexamethasone sodium, methyl prednisolone sodium succinate, and other sodium or potassium salts.

For example, an antidermoinfective drug for topical administration according to the invention is a member of the group consisting of sulfur drugs like sulfamethoxazole sodium; erythromycin and gentamicin sulfate.

For example, a miotic drug for topical administration according to the invention is a member of the group consisting of pilocarpine hydrochloride and carbachol.

For example, an antifungal drug for ophthalmic administration according to the invention is a member of the group consisting of water-soluble salts of amphotericin B, and miconazole.

For example, an antiviral drug for ophthalmic administration according to the invention is a member of the group consisting of acyclovir sodium, ganciclovir sodium, foscarnet sodium and the like.

For example, an anesthetic drug for ophthalmic administration according to the invention is a member of the group consisting of lidocaine hydrochloride, oxybuprocaine hydrochloride, procaine, benzocaine, xylocalne, etidocaine, cocaine, benoxinate, dibucaine hydrochloride, dyclonine hydrochloride, naepaine, phenacaine hydrochloride, piperocaine, proparacaine hydrochloride, tetracaine hydrochloride, hexylcaine, bupivacaine, and mepivacaine.

For example, an antibiotic drug for ophthalmic administration according to the invention is a member of the group consisting of water-soluble salts of amphotericin B, norfloxacin, miconazole nitrate, ofloxacin, idoxuridine, chloramphenicol, colistin sodium methanesulfonate, carbenicillin sodium; beta-lactam antibiotics, cephalosporins like cefoxitin sodium, tetracyclines, neomycin sulfate, carbenicillin sodium, colistin, benzathine penicillin, polymyxin B, vancomycin, chibrorifamycin, gramicidin, bacitracin and sulfonamides.

For example, an aminoglycoside drug for ophthalmic administration according to the invention is a member of the group consisting of gentamycin, kanamycin, amikacin, sisomicin, nalidixic acid analogs such as norfloxacin.

For example, an antibiotic/antiinflammatory combination drug for ophthalmic administration according to the invention is a member of the group consisting of neomycin sulfate and dexamethasone sodium phosphate, timolol maleate and aceclidine.

For example, an antiallergic drug for ophthalmic administration according to the invention is a member of the group consisting of 3′-(1H-tetrazol-5-yl)oxanilic acid(MTCC), ketotifen fumarate and sodium cromoglycate.

For example, an antiinflammatory drug for ophthalmic administration according to the invention is a member of the group consisting of water-soluble salts of cortisone, hydrocortisone, betamethasone, dexamethasone, prednisone, methylprednisolone, medrysone, fluorometholone, prednisolone, and analogs thereof.

For example, an anticholinergic or miotic drug for ophthalmic administration according to the invention is a member of the group consisting of echothiophate, pilocarpine, physostigmine salicylate, diisopropylfluorophosphate, epinephrine, dipivalopylepinephrine, neostigmine, echothiopate iodide, demecarium bromide, carbamoyl choline chloride, methacholine, bethanechol, and analogs thereof.

For example, an antiglaucoma or anticataract drug for ophthalmic administration according to the invention is a member of the group consisting of timolol maleate, carteolol hydrochloride, glutathione, pirenoxine, R-timolol, and a combination of timolol or R-timolol with pilocarpine.

For example, a mydriatic drug for ophthalmic administration according to the invention is a member of the group consisting of atropine, homatropine, scopolamine, hydroxyamphetamine, ephedrine, cocaine, tropicamide, phenylephrine, cyclopentolate, oxybutynin, eucatropine, and analogs thereof.

For example, an antihistamine drug for ophthalmic administration according to the invention is a member of the group consisting of chlorpheniramine maleate and diphenhydramine hydrochloride.

For example, a surgical adjunct therapeutic agent for ophthalmic administration according to the invention is a member of the group consisting of proteases such as alpha-chymotrypsin and dispase and polysaccharide hydrolases such as hyaluronidase.

The invention provides a composition for administration of a therapeutic agent into or delivery of a therapeutic agent to a body cavity of a mammal, such as rectum, urethra, nasal cavity, vagina, auditory meatus, oral cavity or buccal pouch. Any one or more of a wide variety of therapeutic agents are administered or delivered through use of a composition according to the invention. Examples of therapeutic agents for administration or delivery through use of a composition according to the invention are enumerated below:

Analgesics such as tramadol, sodium salicylate, sodium aurothiomate.

Antivirals such as acyclovir sodium;

Anesthetics such as lidocaine, benzocaine, dibucaine, procaine, and xylocalne;

Antifungals such as water-soluble salts of miconazole, econazole, candicidin, and amphotericin B.

Dermatics for purulence such as water-soluble salts of sulfisoxazole, kanamycin, tobramycin and erythromycin;

Antimicrobials such as water soluble salts of beta-lactams, cephalosporins, tetracyclines, polypeptide antibiotics, chloramphenicol, gramicidin, sulfonamides; aminoglycoside antibiotics such as neomycin, netilmicin, streptomycin sulfate, gentamycin, kanamycin, amikacin, sisomicin and tobramycin; nalidixic acid analogs such as norfloxacin and the antimicrobial combination of water soluble salts of fludalanine/pentizidone.

Antibiotic/antiinflammatory combinations such as neomycin sulfate-dexamethasone sodium phosphate;

Anti-glaucoma concomitant therapeutic agents such as timolol maleate-aceclidine;

Anti-pyretics such as sodium salicylate, sodium indomethacin trihydrate, and sodium salicylamide;

Anti-inflammatories such as water-soluble salts of betamethasone, dexamethasone, prednisone, methylprednisolone, medrysone, fluorometholone, fluocortolone, prednisolone and the like;

Miotics such as echothiophate, pilocarpine physostigmine salicylate, diisopropylfluorophosphate, epinephrine, neostigmine, carbachol, methacholine, bethanechol, and dipivolyl epinephraine;

Antihistamines such as pyrilamine, chlorpheniramine, tetrahydrazoline, and diphenhydramine hydrochloride;

Adrenal hormone preparations such as dexamethasone sodium phosphate, water soluble salts of triamcinolone and hydrocortisone;

Adrenergic agonists and/or antagonsists such as epinephrine and an epinephrine complex, or prodrugs such as bitartrate, borate, hydrochloride and dipivefrine derivatives;

Carbonic anhydrase inhibitors such as acetazolamide, dichlorphenamide, 2-(p-hydroxyphenyl)-thio thiophenesulfonamide, 6-hydroxy-2-benzothiazolesulfonamide, and 6-pivaloyloxy-2-benzothiazolesulfonamide;

Muscle relaxants such as succinylcholine chloride, danbrolene, cyclobenzaprine, methocarbomol, and diazepam;

Chelating agents such as ethylenediamine tetraacetate (EDTA) and deferoxamine;

Peptides and proteins such as lutinizing hormone, releasing hormone, vasopressin, interferon, leuprolide acetate and insulin-like growth factor;

Immunosuppressive agents, antineoplastics and anti-metabolites such as adriamycin, cyclophosphamide, methyl prednisolone sodium, hydroxyprogesterone, fluoxymesterone, vinblastine sulfate, vincristine, daunorubicin, doxorubicin hydrochloride, levamisole hydrochloride, hydroxyurea, Ifosfamide, mesna, goserelin acetate, floxuridine, fludarabine phosphate, mitomycin, thiotepa, procarbazine hydrochloride, mechlorethamine hydrochloride, cyclophosphamide, 5-fluorouracil and cytarabine.

The invention thus provides a composition comprising, by mass, from about 1% to about 3% therapeutic agent, from about 0.05% to about 0.5% carbopol, from about 0.1% to about 0.5% hydroxypropylmethylcellulose, from about 14% to about 20% Lutrol F127, from about 13% to about 20% Lutrol F68, from about 0.1% to about 0.5% trolamine 10% w/v aqueous solution, and water.

The invention provides a composition, by mass, 1% clindamycin, 20% poloxamer, and water.

The invention provides a composition comprising, by mass, 1% clindamycin, 0.5% HPMC, 15% poloxamer, and water, The invention provides a composition comprising, by mass, 3% clindamycin, 0.3% carbopol, 15% poloxamer, 0.3% trolamine 10% w/v aqueous solution, and water.

A composition according to the invention is useful for treatment of, therapy of, prophylaxis of, lessening the severity of, amelioration of, or forestalling an injury, a disease, an infection, discomfort, pain, or a malady in a vertebrate.

The invention accordingly provides a method of forestalling infection in a vertebrate, comprising the step of administering to the vertebrate, at a site in or on the vertebrate where it is desired to forestall infection, a therapeutically effective amount of a composition according to the invention.

A composition according to the invention was prepared according to the following formula and found to be useful in the delivery or administration of a therapeutic agent, in this case, clindamycin:

| | |
|---|---|
| Lutrol F127 | 20% |
| Clindamycin | 1% |
| 0.1 M phosphate buffer | qs 100 g |

A further composition according to the invention was prepared according to the following formula and found to be useful in the delivery or administration of a therapeutic agent, in this case, clindamycin:

| | |
|---|---|
| Lutrol F127 | 20% |
| Clindamycin | 1% |
| 0.9% Sodium Chloride in Water | qs 100 g |

Yet a further composition according to the invention was prepared according to the following formula and found to be useful in the delivery or administration of a therapeutic agent, in this case, clindamycin:

| | |
|---|---|
| Lutrol F127 | 15% |
| Carbopol 934F | 0.1-0.5% |
| Clindamycin | 1% |
| Deionized water | qs 100 g |

Preliminary compositions were prepared and tested for the establishment of the properties of said compositions:

| | |
|---|---|
| Lutrol F127 | 15% |
| Lutrol F68 | 18% |
| Deionized water | qs 100 g |
| Lutrol F127 | 15% |
| Lutrol F68 | 18% |
| 0.9% NaCl in Water | qs 100 g |
| Lutrol F127 | 15% |
| Lutrol F68 | 18% |
| 0.1 M Phosphate Buffer pH 7.4 | qs 100 g |

Further preliminary compositions were prepared and tested for the establishment of the properties of said compositions:

| | |
|---|---|
| Lutrol F127 | 15% |
| HPMC | 0.1-0.5% |
| Deionized Water | qs 100 g |
| Lutrol F127 | 15% |
| HPMC | 0.3% |
| Carbomer | 0.3% |
| 0.1 M Phosphate Buffer pH 7.0 | qs 100 g |
| Lutrol F127 | 15% |
| HPMC | 0.3% |
| Carbomer | 0.3% |
| 0.9% NaCl in Water | qs 100 g |
| Lutrol F127 | 15% |
| HPMC | 0.3% |
| Carbomer | 0.3% |
| Deionized Water | qs 100 g |

Further embodiments of the invention are as follows.

Embodiment: Narcotic Analgesics for Sublingual/Buccal and Transdermal Delivery: e.g., Fentanyl.

Solubility: 1000 mg/40 mL=25 mg/mL

Doses: 1.2-1.8 mg

Formulation:

| | |
|---|---|
| Lutrol F127 | 15% |
| Carbopol 934F | 0.1-0.5% |
| Fentanyl | 1% |
| Deionized water | qs 100 g |

Therefore, apply 0.12-0.18 g of formulation to obtain desired dose.

Embodiment: Steroidal Anti-Inflammatory for Topical and Ophthalmic Administration: e.g. Dexamethasone Sodium.

Solubility: 1 g/2 mL Freely Soluble

Doses: 0.05-0.1% Applied Topically

Formulation:

| | |
|---|---|
| Lutrol F127 | 15% |
| Carbopol 934F | 0.1-0.5% |
| Dexamethasone sodium | 0.05-0.1% |
| Deionized water | qs 100 g |

Therefore, apply formulation to obtain desired dose in the eye.

Embodiment: Anti-Viral Agent for Topical: e.g.

Acyclovir Sodium.

Solubility: 1 g/10 mL Water

Dose: 3.0% Topical

Formulation:

| | |
|---|---|
| Lutrol F127 | 15% |
| Carbopol 934F | 0.1-0.5% |
| Acyclovir Sodium | 3.0% |
| Deionized water | qs 100 g |

Embodiment: formulation for delivery of anesthetic: e.g., lidocaine.

Solubility-1 g/l ml of water

Dose—250 mg-350 mg/15 ml

Formulation:

| | |
|---|---|
| Lutrol F127 | 15% |
| Hydroxypropylmethylcellulose | 0.1-0.5% |
| Lidocaine | 3.0% |
| Deionized water | qs 100 g |

Embodiment: formulation for delivery of narcotic analgesics: e.g., morphine sulphate. Formulation:

| | |
|---|---|
| Lutrol F127 | 15% |
| Hydroxypropylmethylcellulose | 0.1-0.5% |
| Morphine sulphate | 3.0% |
| Deionized water | qs 100 g |

Embodiment: formulation for delivery of ophthalmic antibiotic: e.g., ciprofloxacin hydrochloride

Dose—100-200 mg twice daily

Formulation:

| | |
|---|---|
| Lutrol F127 | 15% |
| Hydroxypropylmethylcellulose | 0.1-0.5% |
| Ciprofloxacin lactate | 1.0% |
| Phosphate buffer pH 4.4 | qs 100 g |

Embodiment: formulation for delivery of mydriatic: e.g., atropine sulphate.

Solubility-1 gm/0.5 ml of water

Dose—0.1-0.2 gm

Formulation:

| | |
|---|---|
| Lutrol F127 | 15% |
| Hydroxypropylmethylcellulose | 0.1-0.5% |
| Atropine sulphate | 1.0% |
| Phosphate buffer pH 4.4 | qs 100 g |

The foregoing description and embodiments are merely exemplary and are not intended to limit the scope of the invention, which encompasses all foreseeable and unforeseeable equivalents of what is described herein.

We claim:

1. A composition comprising, by mass, from about 1% to about 3% therapeutic agent, from about 0.05% to about 0.5% carbopol, from about 0.1% to about 0.5% hydroxypropylmethylcellulose, from about 14% to about 20% Lutrol F127, from about 13% to about 20% Lutrol F68, from about 0.1% to about 0.5% trolamine (10% w/v aqueous) solution, and water.

2. A composition comprising, by mass, about 1% clindamycin, about 20% poloxamer, and water.

3. A composition comprising, by mass, about 1% clindamycin, about 0.5% hydroxypropylmethylcellulose, about 15% poloxamer, and water.

4. A composition comprising, by mass, about 3% clindamycin, about 0.3% carbopol, about 15% poloxamer, about 0.3% trolamine about 10% w/v aqueous solution, and water.

5. A method of treatment of, therapy of, prophylaxis of, lessening the severity of, amelioration of, or forestalling an injury, a disease, an infection, discomfort, pain, or a malady in a vertebrate, comprising the step of administering to the vertebrate an effective amount of a composition according to claim 1.

6. A method of forestalling infection in a vertebrate, comprising the step of administering to the vertebrate, at a site in or on the vertebrate where it is desired to forestall infection, a therapeutically effective amount of a composition according to claim 2, claim 3, or claim 4.
